(19) United States
(12) Patent Application Publication
(10) Pub. No.: US 2014/0276807 A1
(43) Pub. Date: Sep. 18, 2014

(54) Spine Fixation Device and Method

(71) Applicant: Laverne Lovell, Memphis, TN (US)

(72) Inventor: Laverne Lovell, Memphis, TN (US)

(21) Appl. No.: 14/217,243

(22) Filed: Mar. 17, 2014

Related U.S. Application Data
(60) Provisional application No. 61/801,809, filed on Mar. 15, 2013.

(51) Int. Cl. A61B 17/70; A61B 18/08

(52) U.S. Cl. CPC A61B 17/7074; A61B 18/082
USPC 606/45

(57) ABSTRACT

A novel device and method for surgical inducement of cervical vertebra fusion as a treatment for cervical spinal disease using a guide wire, slide knife, and a minimally invasive portal access device and an inter-vertebra linking member.

### BACKGROUND OF THE INVENTION

This invention relates generally to devices and method for surgically immobilizing or fusing two or more human vertebral bodies. More particularly, it relates to a minimally invasive surgical technique allowing for a linear posterior approach to purchasing one or more within a vertebrae and placement of plates, rods or other such hardware to enable proper bone fusion between a plurality of vertebrae.

Normally, two adjacent vertebra would usually move relative to one another creating a motion segment unit. While each motion segment unit may only move a few degrees, the collective movement of all the vertebral motion segment units allows for great flexibility of the spine. When the motion segment unit becomes diseased, a patient may experience localized or radiating pain or even paralysis necessitating treatment. Treatment of cervical spinal disease, such as disc degeneration, disc herniation, instability, spinal stenosis, spondylosis, and facet joint arthritis frequently requires the fusion of two or more adjacent cervical vertebrae. Current techniques utilize both anterior and posterior approaches for fusion, depending on the pathology present at the time of initial surgery. Failure to fuse after anterior approach results in chronic pain and necessitates repeat surgery to secure fusion and relieve this pain. This repeat surgical fusion can be done anteriorly, with increased risk of injury to vital neck structures, or be done posteriorly with a reduced risk of injury to vital neck structures.

A common posterior operative approach to achieve cervical joint fusion involves proper placement of a plurality of bone screws within a cervical lateral mass. A screw placed in one lateral mass is connected to a screw placed in an adjacent lateral mass. The space between the lateral masses may be packed with bone, or other bone growth promoting substance. The completed construct, ideally, reduces motion sufficiently so that proper bone fusion occurs between the adjacent cervical vertebrae. For proper screw purchase, the screws are ideally placed at an angle to the vertical axis of the spine thereby requiring one or more lengthy incisions, substantial muscle dissection and severe postoperative pain and an extended recovery.

A need exists for a surgical apparatus enabling a posterior approach that minimizes incision length and muscle dissection thereby reducing the operative risk to patient, blood loss, postoperative pain, narcotic consumption, time to return to normal activities and work, operating room time/cost, and reducing hospital stay and cost.

### BRIEF SUMMARY OF THE INVENTION

An embodiment of the invention disclosed herein comprises of an electrocautery sliding knife, referred herein to as “ESK,” and a minimally invasive portal access device, referred herein to as “MIPAD,” enabling precise placement and purchase of a plurality of bone screws into a plurality of cervical vertebrae enabling additional hardware, such as one or more posterior cervical plates, to immobilize the vertebral bodies relative to one another.

The Electrocautery Sliding Knife is a new device enabling a novel technique whereby sliding a combined cauterizing and cutting edge down a previously placed guide rod. The guide rod, also referred to herein also as a “Docking Rod”, described in further detail below, is purchased in the cervical facet joint. The Electrocautery Sliding Knife creates a minimally invasive portal for access to the posterior lateral mass of the cervical spine. This portal receives the MIPAD, also described in further detail below, facilitating the stabilization and fusion. These devices help to eliminate the long skin incision and painful muscle dissection otherwise needed in current posterior cervical approaches.

The Minimally Invasive Portal Access Device has multiple functions in the process of the posterior cervical lateral mass stabilization and fusion technique. In the embodiment, MIPAD is shaped to conform to the opening created by the ESK and angled at the distal end to lie flat against the lateral cervical spine. The distal end of the embodiment of the MIPAD will have attached to it the plate that will, along with anchoring screws, fixate a lateral mass of a first vertebra to a lateral mass of the adjacent vertebra.

In the embodiment, the MIPAD has three elongated apertures in the form of incorporated tubes running the length of the MIPAD. Each tube has a removable cannula in its center. The center tube is the guide tube for initial insertion, stabilization for drilling and screw placement, and after removal of its cannula, for final placement of bone grafting material into the facet joint. The center tube cannula is also threaded at the distal end for plate fixation for initial placement against the lateral masses. The two other tubes of the embodiment, the rostral and caudal tubes, will be access tubes to drill into the lateral masses and, after removal of their cannulas, placement of stabilizing screws to anchor the attached plate.

Lateral Mass Cervical Plate (Plate) is, in the embodiment, approximately 25 mm long 6 mm wide plate is designed for fixation of the posterior lateral masses of the cervical spine. The plate has three holes laterally aligned. The end holes receive the fixation screws for tightly securing it to bone. The middle hole is present for initial passage over the Docking Rod, and after removal of the middle cannula from the MIPAD provides an opening to drill into the facet joint for placement of bone/bone substitute for fusion.

This plate is angled 10 degrees to provide flat fixation to the lateral masses and to receive the MIPAD at 10 degrees facilitating proper screw angle. The holes in the plate are drilled with a 40 degree rostral-caudal and a 10 degree medial-lateral angle to achieve proper screw placement in the typical cervical lateral mass.

A surgical method for posterior cervical lateral mass fusion using the ESK and MIPAD system begins with fluoroscopic visualization of the cervical spine in the anesthetized intubated prone patient used to identify skin surface landmarks in order to select the appropriate entry point and angle to engage the facet joint with a K-wire. Anterior-Posterior (AP) and lateral fluoroscopy will be needed for this placement and used as deemed necessary by the surgeon throughout the remainder of the procedure.

The K-wire is then inserted starting at the selected entry point and ending with a 4-5 mm insertion into the desired facet joint. A cannulated approximately 2 mm diameter Docking Rod with cutting threads on the distal end is then placed over the wire and screwed into the facet joint for stabilization during the case. The K-wire is then removed.

The ESK is then used to create the minimally invasive portal from the posterior para-midline entry through the skin and posterior spinal muscle mass, ending by abutting up against the cervical spine lateral masses. The ESK slides down the previously placed Docking Rod to create this portal.

The ESK is removed leaving the Docking Rod in place. The MIPAD with plate attached is inserted over the Docking Rod and into the soft tissue portal created by the ESK.

At this point during the method, the MIPAD serves as the plate holder up against the boney spine, and also as a channel guide for insertion of drill bit through the rostral cannula and 10 mm deep into the lateral mass. After creation of this hole, the drill bit and inner cannula are removed. Through the remaining tube a 14 mm screw is placed to fixate the top of the plate to the spine.

The caudal cannula is now accessed with a drill bit to create a 10 mm hole in the lower lateral mass. The cannula is then removed with the drill bit and a second screw is inserted to fixate the bottom of the plate to the spine. The plate is now firmly fixed to the spine with stabilization of the lateral masses to one another.

In this embodiment, the Docking Rod in the facet joint is then removed along with the center cannula from the MIPAD, leaving the MIPAD in place. Through the center MIPAD tube a larger drill is used to enter the facet joint and abraid the cartilaginous endplates. Placement of a threaded bone dowel, bone morphogenetic protein, or other bone substitute can then performed to facilitate fusion of this joint.

The MIPAD is then removed and xray used for final confirmation of acceptable plate and screw placement.

The above procedure is then repeated if fixation is desired on the other side of the spine.

### BRIEF DESCRIPTION OF THE DRAWINGS

An embodiment of the invention is illustrated by the accompanying drawings, in which:

FIG. 1 is a front perspective view of the posterior of a cervical spine showing the implantation of two posterior cervical plates.

FIG. 2 is a back view a posterior cervical plate.

FIG. 3 is a lateral view of the posterior cervical plate.

FIG. 4 is a top view of the posterior cervical plate.

FIG. 5 is a section view of the posterior cervical plate taken on line 5-5 in FIG. 2.

FIG. 6 is a section view of the posterior cervical plate taken on line 6-6 in FIG. 2.

FIG. 7 is a perspective view of the posterior cervical plate in a plane perpendicular to the axes of the plate's apertures.

FIG. 8 is a lateral view of the plate, the minimally invasive portal access device and cannulas.

FIG. 9 is a lateral view of the cannulas

FIG. 10 is a lateral view of the minimally invasive portal access device.

FIG. 11 is a section view of the minimally invasive portal access device taken on line 11-11 of FIG. 10.

FIG. 12 is a section view of the minimally invasive portal access device taken on line 12-12 of FIG. 10.

FIG. 13 is a top view of the plate, minimally invasive portal access device and docked cannulas.

FIG. 14 is a lateral view of the plate, minimally invasive portal access device and docked cannulas.

FIG. 15 is a lateral view of the slide knife.

FIG. 16 is a top view of the slide knife.

FIG. 17 is a perpective view of the slide knife taken on a plane perpendicular to the axis of the cannulated aperture.

FIG. 18 is a lateral view of a cervical spine with a docking rod inserted into the left facet joint.

FIG. 19 is a posterior view of a cervical spine with a docking rod inserted into the left facet joint.

FIG. 20 is a lateral view of the slide knife ready to create a surgical portal.

FIG. 21 is a lateral view of the slide knife creating the surgical portal.

FIG. 22 is a lateral view of the minimally invasive portal access device ready to descend the surgical portal.

FIG. 23 is lateral view of the minimally invasive portal access device within the surgical portal.

FIG. 24A is a lateral view of drilling a first hole into the lateral mass of the upper vertebra.

FIG. 24B is a lateral view of insertion of a screw into the lateral mass of the upper vertebra.

FIG. 25 is a lateral view of drilling a second hole into the lateral mass of the lower vertebra.

FIG. 26 is a lateral view of insertion of a screw into the lateral mass of the lower vertebra.

FIG. 27 is a lateral view of removal of the docking rod.

FIG. 28 is a lateral view of removal of the second cannula and release of the minimally invasive portal access device from the plate.

FIG. 29 is a lateral view of the completed plate implantation.

FIG. 30 is a posterior view of the completed plate implantation.

FIG. 31 is a top view of an ostetome.

FIG. 32 is a lateral view of an ostetome.

FIG. 33 is a perspective view of a bifurcated docking rod.

FIG. 34 is a lateral view of an alternative slide knife.

FIG. 35 is a front view of an alternative slide knife.

### DETAILED DESCRIPTION OF THE INVENTION

The drawings illustrate a novel device and method for surgical inducement of cervical vertebra fusion as a treatment for cervical spinal disease.

FIG. 1 shows a perspective view of the first embodiment of the present invention 1 positioned upon a cervical column of a typical patient. Skin, muscles, ligaments and blood are not illustrated in this figure for clarity and ease of understanding. The lateral mass cervical plate 101 positioned on the left is positioned upon the left posterior portion lateral masses 15, 35 of the upper and lower cervical vertebrae 11, 31 of the motion segment unit which is targeted for fusion. The plate 101, acts as an inter-vertebra linking member, rigidly connecting upper vertebra to the lower vertebra. One plate 101 may be used on either the left or right side of the vertebras, or as shown in FIG. 1, two plates 101 may be used. In the embodiment, the plate is 25 mm long by 5 mm wide. Each plate 101 possesses three apertures 135, 145, 155 extending from the plate front surface 103 to the plate back surface 105. Each aperture is aligned laterally. The first or top aperture 135 and the third or bottom aperture 155 each receive fixation screws 171, 181 for tightly securing the plate to the vertebral bone. The second or middle aperture 145 allows the plate 101 to be passed over a docking rod, such as a guide wire or k-wire, and after removal of the middle cannula from the MIPAD, provides an opening to drill into the facet joint and, if desired, placement of bone and/or bone substitute to aid in bone fusion.

FIG. 2 shows a front view of a plate 101 to be positioned upon the right side of the vertebra showing the front surface 103, the top surface 107, and the front edge of the medial side surface 113, the front edge of the distal side surface 111 and the front edge of the bottom surface 109. While this figure, and each subsequent figure of the plate 101, shows the right side plate, the left side plate should be understood to be a mirror image. The medial side surface 113 and distal side surface 111 each have a width extending from the back surface 105 to the front surface 103. In the embodiment, the medial lateral side surface 113 width is shorter than the width of the distal lateral side surface 111, resulting in an angled front surface 103. The angled front surface 103 allows the proper positioning of the plate upon the vertebral bones with the MIPAD. Each aperture 135, 145 and 155 possesses a circular cross section, when viewed perpendicular to the central axis 137, 147, 157 of each aperture. The central axis 137, 147, 157 of each aperture is angled upward as it passes from the front surface 103 to the back surface 105 of the plate 101 and at a right angle to the front surface when the implant is viewed from the top, angling the aperture toward the medial side surface 113 as it passes from the front surface 103 to the rear surface 105 of the plate 101. A ledge 139, 149, 159 is present in each aperture, formed by a slightly larger diameter of the aperture near the front surface 103 to provide a seating surface for the screw head.

FIG. 3 shows a medial side view of the plate 101 showing the front surface 103, medial side surface 113 and a portion of the top surface 107 and a portion of the bottom surface 109. The front portion of each aperture 135, 145, 155 is visible as well as the ledge 139, 149, 159 present within each aperture. The top side and bottom slide are angled downward from the back side of the plate to the front side.

FIG. 4 shows a top view of the plate, showing the top surface 107, the top edge of the front surface 103 and the top edge of the rear surface 105. The top surface 103 is positioned at an angle to the bottom surface 105, allowing for proper alignment of the implantation instrument(s) and seating of the plate back surface 105 on the vertebral bone.

FIG. 5 shows a section view of the plate 101 taken on line 5-5 of FIG. 2 showing the inner surface of the second aperture 145. The aperture ledge 145 provides a recess for the bone screw head. The aperture central axis 147 is observed to be at a right angle to the front surface 103 of the plate 101 when viewed from the top as this figure is shown. The angle of the front surface 103 and aperture central axis 147 may be selected and vary slightly from what is shown in the embodiment depending upon the desired alignment and/or the contours of the patient's vertebra.

FIG. 6 shows a section view of the plate 101 taken on line 6-6 of FIG. 2 showing the inner surfaces of the first second and third apertures 135, 145, 155 respectively. The angle of the aperture central axis 137, 147, 157 is set for any given plate; however different embodiments of the invention may place the angle of the aperture at a different angle depending upon the angle of the facet joints of the lateral masses of the particular patient being operated upon. The downward swept angle from the back surface 105 to the front surface 103 allows the purchase of a bone screw without damaging the facet joints adjacent of the motion segment unit being fused.

FIG. 7 shows a perspective top view, viewed from an angle normal to the front surface 103 of the plate 101. The three apertures 135, 145 and 155 and corresponding ledges 139, 149, 159 visible.

FIG. 8 shows an exploded view of an embodiment of the invention, including the plate 101, Minimally Invasive Portal Access Device, or MIPAD, 201 and cannulas 301, 303, 305. The first cannula 301 and third cannula 305 narrow the first aperture 203 and third aperture 263 of the MIPAD 201. An alternative embodiment allows the cannula to enter into the first and third aperture of the plate 101 providing additional rotational stabilization of the plate 101 when assembled to the MIPAD 201.

FIG. 9 shows the first, second, and third cannulas 301, 331, 361 that are removably inserted into the first, second, and third apertures 203, 233, 263, also referred herein to as aperture tubes, of the embodiment of the MIPAD. The end 333 of the second, or middle, cannula 331 possesses threads 335 to engage the second aperture 145 of the plate 103, allowing retention of the plate on the end of the MIPAD 201 allowing the plate 101 to be properly positioned against the lateral masses of the vertebra selected for fusion. The rostral, or first, aperture tube 203 and the caudal, or third, aperture tube 263 allow access to drill into the lateral masses, and after removal of the cannulas 301, 331, 361, allow for placement of stabilizing screws to anchor the attached plate 101 to vertebral bone.

FIG. 10 shows a left side view of the MIPAD 201. The left surface 221, left edge of the front surface 225 and left edge of the back surface 227 is shown. The angle formed between the front surface 225 and the top surface 229 and between the front surface 225 and bottom surface 231 may be varied depending upon the angle of patient's facet joint and lateral mass surface.

FIG. 11 shows a section view of the MIPAD 201 taken on line 11-11 of FIG. 10. The first, second and third cannulas 135, 145, 155 are circular in shape, centered within the MIPAD 201 cross-section.

FIG. 12 shows a section view of the MIPAD 201 taken on line 12-12 of FIG. 10.

FIG. 13 shows a top view of the embodiment of the invention 1, the plate 101, the front surface 225 of the MIPAD 201, and cannula 301.

FIG. 14 shows a left side view of the embodiment of the invention 1, the plate 101, the front surface 225 of the MIPAD 201, and first, second and third cannula 301, 331, 361. The threads 335 of the second cannula 331 protrude past the back surface 227 of the MIPAD 201 to engage corresponding threads within the second aperture of the plate 101.

FIG. 15 shows the embodiment of the cauterizing slide knife 401. The slide knife 401 comprises of a shaft 411 and a blade 421. The shaft 411 is hollow allowing a docking rod, such as a k-wire, to run down its length. The blade 411 has a cutting edge 441 along the front edge 423 of the blade. The front edge 423 and shaft 411 are positioned at an angle corresponding to the angle of patient's facet joint and lateral mass surface.

Electrocauterization enables the slide knife to make an opening through soft tissue to allow the physician to operate without excess bleeding of the patient. The cauterization occurs by passing a current through the leading cutting edge 441 of the blade. The cutting edge 441, or a portion thereof possesses an electrically conductive surface 443. The remaining surfaces of the blade 421 are nonconductive to prevent excess current drain and excessive damage to the surrounding tissues. The nonconductive surface properties may be achieved by constructing the majority of the blade from a non-conductive material, or coating the blade with a non-conductive material, preferably a nonconductive ceramic, plastic, rubber or other polymer material.

A top view of the cauterizing slide knife 401 as shown in FIG. 16 reveals the tapered cutting edge 419 of the front edge 413 of the shaft 411. A cauterization handle may be attached to the back end 415 of the shaft 411 to provide a comfortable grip for control of the instrument by the physician.

FIG. 17 shows a front view along the shaft 411 of the slide knife 401. Cutting occurs along the cutting edge 441 of the blade 421 and front cutting edge 419 of the shaft 411. A hollow aperture 451 runs the length of the shaft 411 allowing the shaft to slide over and along a docking rod imbedded into the facet joint of the level to be fused.

Once the angles of the facets are measured, the spine fusion procedure begins with a patient lying prone with the head and neck aligned with the body. The entry point is marked on the patient's skin on the posterior of the patients neck. FIG. 18 shows the surface of the patient's skin 51 as a dotted line, and a portion of the patient's cervical vertebra 11, 31. Other soft tissue, bones, and fluids have been omitted for clarity of illustration. A docking rod 471 is passed, as shown in FIG. 18, from the outer surface of the patients skin 51 and down to the facet joint of the joint that is to be fused. The docking rod, preferably having a threaded end, is imbedded in the facet joint 21. The positioning of the docking rod may be assisted by computer guidance, radiographs, fluoroscope, or other such imaging devices to ensure a proper approach angle to the facet and verify the correct positioning, both from anterior-posterior and lateral points of view. Here the left side of the cervical joint is being fused. It should be understood that the procedure may be mirrored for the right side of the spine in addition to other vertebral levels than those illustrated here.

Alternatively, after the guide wire is inserted starting at the selected entry point and ending with a 4-5 mm insertion into the desired facet joint, a cannulated, approximately 2 mm diameter, docking rod with cutting threads on the distal end is then placed over the wire and screwed into the facet joint for stabilization. The guide wire may then be removed, leaving the cannulated docking rod purchased in the facet joint.

FIG. 19 shows a posterior partial view of the spine and vertebra 11, 31 to be fused. Other tissues, bones and fluids have been omitted from the illustration for clarity. The docking rod 471 passes into and is purchased within the left facet joint 21 at the joint to be fused. The docking rod is angled, preferably 5-15 degrees medially.

Once the docking rod is placed, the slide knife 401 is placed over the docking rod 471. In the embodiment, the docking rod is constructed from a non-conductive material to prevent shorting, however, a nonconductive coating may be placed over the docking rod, or a sheath may be placed down the docking rod prior to placing the slide knife down the guide to prevent shorting the cauterization function of the slide knife. Alternatively, a stiffer non-conductive cannulated docking rod may be placed down around the docking rod, then once the cannulated docking rod is purchased in the facet joint, the flexible docking rod may be removed prior to utilization of the slide knife.

FIG. 20 shows a lateral view of the slide knife 401 prepared to make a longitudinal incision in the patient's back down to the operative level of the cervical spine vertebrae 11, 31 to be fused. A cauterization handle (not shown) may be attached to the shaft 411 of the slide knife 401.

FIG. 21 shows the slide knife 401, attached to a cautery handle 461, cutting a surgical portal 61 from the surface of the patient's skin 51 to the posterior surface of the lateral masses 15, 35. The surgical portal height is determined by the height of the cutting blade 421, the top edge of the portal 63 and the lower edge of the portal 65 as illustrated in FIG. 21. At this point the physician may prepare the bone surface for the plate. Soft tissues may be scraped away using an osteotome.

After the portal is created, the slide knife 401 is removed and the Minimally Invasive Portal Access Device 201 second aperture 233 is slid down around the docking rod 471 as shown in FIG. 22.

The MIPAD 201, assembled with the fusion plate 101 is slid down the docking rod 471 until resting upon the rear surface of the lateral masses 15, 35 of the upper and lower cervical vertebra 11, 31 to be fused as shown in FIG. 23. The MIPAD 201, acts as a sleeve maintaining the surgical portal opening 61, minimizing trauma to the surrounding tissues and bleeding while maintaining surgical access to the lateral masses. A secondary sleeve may be slid down over the MIPAD allowing removal of the MIPAD while maintaining the portal opening.

FIG. 24A illustrates the MIPAD 201 and plate 101 in place over the docking rod 471. While aligned with the lateral mass 15 of the cervical body 11 the surgeon slides the drill bit 281 down the first cannula 301 and drills into the lateral mass 15 until the tip 283 of the drill bit 281 is of a desired depth.

In FIG. 24B the drill bit 281 is removed from the cannula 301, the cannula 301 is removed from the MIPAD 201 and a screw 171 may then be slid down the first aperture 203 and purchased using a torque driver 191 into the lateral mass body 15.

FIG. 25 shows the drilling of the lateral mass body 35. The drill bit 281 is slid down the third cannula 361 and drills into the lateral mass 15 until the tip 283 of the drill bit is of a desired depth. The drill bit 281 is removed from the cannula 361, the cannula 361 is removed from the MIPAD 201 and a screw may then be slid down the third aperture 233 and purchased into the lateral mass body 15.

FIG. 26 shows the MIPAD 201 with the first cannula 301 and third cannula 361 removed. The lower screw 181 secures the lower half of the plate 101 to the lateral mass 35 of the lower vertebra 31. The upper screw 171 attached to a torque driver 191 prepares to slide down the first aperture 203 of the MIPAD 201 and secure the upper half of the plate 101 to the lateral mass 15 of the upper vertebra 11.

Once the screws 171, 181 and plate 101 are secure, the docking rod 471 may be removed from the facet joint 21 and second cannula 331 as shown in FIG. 27.

The second cannula 331 is then unscrewed from the plate 101 as shown in FIG. 28. In the embodiment of the procedure, the second cannula 331 is removed from the MIPAD 201 and a drill bit is inserted and used to remove bone and cartilage from the facet joint to create a cavity. A bone graft, or bone graft substitute is then preferably placed within the cavity. The MIPAD 201 is then removed from the portal 61.

The plate 101 is secured to the vertebra lateral masses 15, 35 as shown in FIG. 29. The portal 61 may now be surgically closed. The surgical process may then be repeated for the right side of the patient, securing a second posterior fixation plate.

FIG. 30 shows a posterior view of the plate 101 secured to the left lateral masses 15, 35 of the upper vertebra 11 and lower vertebra 31.

During the procedure, prior to placing the plate and after the portal is opened, it may be necessary to prepare the lateral mass surface by scraping the bone with an osteotome. FIG. 31 shows a top view of a an osteotome 501 having a plurality of lateral cutting edges 541, 543 along the anterior surface 533 edges. The blade 531 anterior edge 533 is angled relative to the shaft 511 and handle 521 to allow the tool to easily access the bone through the portal 61. The shaft 551 and blade 531 of the osteotome are cannulated allowing them to pass over the docking rod. A knurled handle 521 provides the physician with ample control of the instrument allowing the instrument to be rotated within the surgical portal 61.

Multiple vertebral levels may be fused by stepped alternating side unilateral plating using the above technique. For example, C4, C5, and C6 vertebral levels may be fused by implantation of a plate between the left lateral masses of C4 and C5, and along the right vertebral masses of C5 and C6.

In a third embodiment of the invention, a docking rod 601 is bifurcated 631 as shown in FIG. 33 from its posterior end 613 to its threaded end 611. In the third embodiment, the electrocautery slide knife blade 721, FIG. 34, 35, is then slid between the bifurcation 631 of the docking rod 601 when the docking rod is purchased in the facet joint. The cannulated shaft 711 of the slide knife 701 slides over the bifurcated docking rod 601. As the slide knife 701 slides down, it creates an operating portal while being guided by the docking rod 601.

What is claimed is:

1. An apparatus for the minimally invasive surgical implantation of instrumentation to aid in bone fusion of two adjacent vertebra, the apparatus comprising:
a docking rod suitable to be posteriorly purchased within and parallel to the facet joint of said two adjacent vertebra;
a cauterizing slide knife possessing a cannulated shaft and a blade having a cutting edge;
a cannulated minimally invasive portal access device, said minimally invasive portal access device having a maximum diameter smaller than the height said cauterizing slide knife; and
an inter-vertebra linking member, said inter-vertebra linking member being releasably attached to said cannulated surgical minimally invasive portal access device;
wherein when said docking rod is posteriorly purchased within and parallel to said facet joint of said two adjacent vertebra and said cannulated shaft of said cauterizing slide knife is placed over said docking rod, said cutting edge of said blade is approximately parallel with the posterior surface of said two adjacent vertebras' lateral masses.

2. The apparatus of claim 1 wherein said cannulated minimally invasive portal access device further comprises:
a first aperture;
a second aperture;
a third aperture;
a first cannula having an outer diameter sized to releasably fit within said first aperture;
a second cannula having an outer diameter sized to releasably fit within said second aperture, said second cannula having a threaded end sized to releasably engage a set of threads upon said inter-vertebra linking member;
a third cannula sized to releasably fit within said third aperture.

3. The apparatus of claim 2 wherein said docking rod is cannulated.

4. The apparatus of claim 2 wherein said docking rod is bifurcated.

5. An apparatus for the minimally invasive surgical implantation of instrumentation to aid in bone fusion of two adjacent vertebra, the apparatus comprising:
a cannulated docking rod suitable to be posteriorly purchased within and parallel to the facet joint of said two adjacent vertebra, said docking rod able to be inserted over a guide wire;
a cauterizing slide knife possessing a cannulated shaft and a blade having a cutting edge;
a cannulated minimally invasive portal access device, said minimally invasive portal access device having a maximum diameter smaller than the height said cauterizing slide knife; and
an inter-vertebra linking member, said inter-vertebra linking member being releasably attached to said cannulated surgical minimally invasive portal access device;
wherein when said docking rod is posteriorly purchased within and parallel to said facet joint of said two adjacent vertebra and said cannulated shaft of said cauterizing slide knife is placed over said docking rod, said cutting edge of said blade is approximately parallel with the posterior surface of said two adjacent vertebras' lateral masses.

6. A method for the minimally invasive surgical implantation of instrumentation to aid in bone fusion of two adjacent vertebra, the method comprising:
purchasing a docking rod within and parallel to the facet joint of said two adjacent vertebra;
sliding a cauterizing slide knife down said docking rod, said cauterizing slide knife being guided by said docking rod, thereby creating an operating portal;
sliding a cannulated minimally invasive portal access device over said docking rod, and down within said operating portal; and
fastening an inter-vertebra linking member, said inter-vertebra linking member being releasably attached to said cannulated surgical minimally invasive portal access device.
